SEQUENCE LISTING (8 sequences)

SEQ ID NO: 1 - DNA, 67 bp, Artificial Sequence
  primer for His-tag RNA aptamer
gccagctcccggggccaatcccaaccagaccacccatagccccccctatagtgagtcgtattagtcc

SEQ ID NO: 2 - RNA, 40 nt, Artificial Sequence
  His-tag RNA aptamer
gcuauggguggucugguugggauuggccccgggagcuggc

SEQ ID NO: 3 - DNA, 59 bp, Artificial Sequence
  primer for mini-HER2 RNA aptamer
agccgcgccctaccctatccctcccctcgcggctcccctatagtgagtcgtattagtcc

SEQ ID NO: 4 - DNA, 106 bp, Artificial Sequence
  primer for full-length HER2 RNA aptamer
gatccccgcggaagcttcattttcttgactagtcgtgccctaccctatccctcccctcacaacttttggtaccacgcgtcccccctatagtgagtcgtattagtcc

SEQ ID NO: 5 - RNA, 34 nt, Artificial Sequence
  mini-HER2 RNA aptamer
agccgcgaggggagggauaggguagggcgcggcu

SEQ ID NO: 6 - RNA, 82 nt, Artificial Sequence
  full-length HER2 RNA aptamer
gggacgcgugguaccaaaaguugugaggggagggauaggguagggcacgacuagucaagaaaaugaagcuuccgcggggauc

SEQ ID NO: 7 - DNA, 95 bp, Artificial Sequence
  primer for PSMA RNA aptamer
tcgggcgagtcgtctgccgatgaggattgacaaggagtgacgtaaacatggctgatccgcatcgtcctcccccctatagtgagtcgtattagtcc

SEQ ID NO: 8 - RNA, 71 nt, Artificial Sequence
  PSMA RNA aptamer
gggaggacgaugcggaucagccauguuuacgucacuccuugucaauccucaucggcagacgacucgcccga

(19) United States
(12) Patent Application Publication
(10) Pub. No.: US 2015/0017662 A1
(43) Pub. Date: Jan. 15, 2015

(54) METHOD FOR DETECTING AND QUANTIFYING A TARGET PROTEIN OR A TARGET CELL USING AN APTAMER CHIP

(71) Applicant: University-Industry Cooperation Group Of Kyung Hee University, Gyeonggi-do (KR)

(72) Inventors: Sang Soo Hah, Gyeonggi-do (KR); Gwan Ho Lee, Gyeongsangbuk-do (KR); Ji Su Kim, Incheon (KR)

(73) Assignee: University-Industry Cooperation Group fo Kyung Hee University, Gyeonggi-do9 (KR)

(21) Appl. No.: 14/375,079

(22) PCT Filed: Jan. 14, 2013
(86) PCT No.: PCT/KR2013/000287

§ 371 Date: Jul. 28, 2014

(30) Foreign Application Priority Data
Jan. 27, 2012 (KR) 10-2012-0008482
Oct. 9, 2012 (KR) 10-2012-0111999

(51) Int. Cl. G01N 33/574; G01N 33/543

(52) U.S. Cl. CPC G01N 33/57492; G01N 33/54306
USPC 435/7.23

(57) ABSTRACT

The present invention is a method for detecting or quantifying a target cell or a target protein using an aptamer chip, and particularly, in order to detect a target cell, a method for detecting and/or quantifying a target cell by reacting a cell staining solution (e.g. 4′,6-diamidino-2-phenylindole (DAPI)) with an aptamer chip which the target cell is bound, in order to detect a target protein, a method for detecting and/or quantifying a target protein by reacting Coomassie Brilliant Blue solution with an aptamer chip which the target protein is bound, and a method for reusing the aptamer chip, wherein the aptamer chip comprises a board to which the aptamer specifically combining with the target cell or the target protein is bonded by a disulfide bond.

### TECHNICAL FIELD

The present invention is a method for detecting or quantifying a target cell or a target protein using an aptamer chip, and particularly, in order to detect a target cell, a method for detecting and/or quantifying a target cell by reacting a cell staining solution (e.g. 4′,6-diamidino-2-phenylindole (DAPI)) with an aptamer chip which the target cell is bound, in order to detect a target protein, a method for detecting and/or quantifying a target protein by reacting Coomassie Brilliant Blue solution with an aptamer chip which the target protein is bound, and a method for reusing the aptamer chip, wherein the aptamer chip comprises a board to which the aptamer specifically combining with the target cell or the target protein is bonded by a disulfide bond.

### BACKGROUND ART

An aptamer in itself has a stable tertiary structure and is a special single-stranded nucleic acid (DNA, RNA or variant nucleic acid) which can be bound to the target molecule with high affinity and specificity.

An aptamer can be obtained by random sequence nucleic acid libraries, SELEX (Systematic Evolution of Ligands by Exponential Enrichment).

Such an aptamer is considered to be a good alternative for antibodies, and many aptamers have been known to bind specifically to small chemical molecules such as metal ions, proteins and even cells with the level of nano- or pico-molar dissociation constants. In addition, an aptamer has properties such as those that follow, according to detailed experiments, which are more favorable when compared with those of antibodies. The first property is that an aptamer can be obtained from nucleic acid libraries built to target molecules (from small inorganic ions to cells). The properties overcome the limitations of antibodies which have to be obtained from cells or animals. The second property is that the selected aptamer can be amplified through PCR (Polymerase chain reaction) or a transcription process can be performed in order to obtain many aptamers with high purity. The third property is that a functional group of an aptamer is easily modified when the aptamer is used for another purpose such as binding on a solid surface because an aptamer has a simple chemical structure. Finally, an aptamer can be used for chemical applications requiring harsh conditions (high temperature or extreme pH) because an aptamer is much more stable than antibodies. Additionally, an aptamer has characteristics which are economical because the aptamer can be chemically synthesized in great quantity, has an affinity to a target coming close to antibodies, and is much smaller in size (by about 1˜2 nm) than antibodies.

Research of a biosensor specific to a molecule using an aptamer has been ongoing due to the effects of aptamers which are superior to those of antibodies. Particularly, manufacturing an aptamer array is very useful. Recently, high-density DNA-based microarrays, which are in the spotlight as a powerful tool for analyzing genes and diseases, have been used for the detection of nucleic acids such as DNA, but the detection results alone do not provide all of the necessary information and thus, in order for protein detection and quantification of more information, the need for nucleic acid type aptamer-based microarrays has been growing. To get information on a specific protein or a cell expressing a specific protein on the surface, a microarray is often used, which is based on proteins immobilized on a solid surface and the proteins may be antigens, antibodies, ligands or receptors specifically binding to the above protein. However, the microarray is economically low in terms of usage of the microarray after fixing antibodies on the surface, and is not easily maintained in a constant form without protein denaturation. In this respect, an aptamer-based microarray can be potentially expanded to a DNA-based microarray in order to recognize cells or expressed proteins. An aptamer is a nucleic acid and thus an aptamer-based microarray easily detects a target material as well as quantifies the material. For development of the aptamer-based microarray, research for developing a very small aptamer-based microarray is ongoing by applying the experience gained from DNA arrays.

A typical biosensor consists of two components in general. To be more specific, one is a component (enzyme, antibody, acceptor, and so on) that is able to recognize a target, and the other is a transducer. A biosensor is an analysis tool requiring relatively simple processes, compared to other analysis tools like spectrophotometers or mass spectrometers requiring several separate steps of analysis to analyze one material. When an aptamer is applied to a biosensor, the aptamer recognizes a target material with high affinity and the properties of the aptamer are easily adjustable. However, because the sensitivity of a biosensor is significantly affected by the transducer, many transducers have been developed in order to be applied to an aptamer-based biosensor for detection and quantification of a specific molecule. Nevertheless, transducing techniques for converting the signal generated by a combination of the developed aptamer and biomaterials are a type of electrochemical signal detection (see Korean Laid-open Patent Publication No. 10-2011-0126942), or fluorescence detection (see Korean Patent No. 10-0896987). The techniques have the limitation of micro to nano-molar level detection, and expensive equipment or chemicals are required for detection and quantification in applying the techniques. Moreover, for detection and quantification using electrochemical signals or fluorescence, a process, in which chemicals capable of oxidizing or reducing, or fluorophores are attached to a target protein, is required and the analysis is done through several steps.

### DISCLOSURE

### Technical Problem

The present inventors spent effort to overcome the inefficiency and problems of existing biosensors requiring expensive equipment. Therefore, the inventors developed the present invention from the finding that it can seen by the naked eye that the intensity of the signal increases in proportion to the number of target cells or the target protein concentration without additional equipment or with a simple process such as being exposed to UV, that a common analyzer can be used for quantification analysis of the target protein, and that the chip can be reused by reducing disulfide bond, if an aptamer-based biochip is analyzed by Coomassie Brilliant Blue staining or cell staining.

### Technical Solution

A purpose of the present invention is to provide a method for detecting a target cell using an aptamer chip, comprising (a) bringing a sample comprising a target cell into contact with an aptamer chip; and (b) reacting a cell staining solution with an aptamer chip bound to the target cell.

Another purpose of the present invention is to provide a method for quantifying a target cell using an aptamer chip, comprising (a) bringing a sample comprising a target cell into contact with an aptamer chip; (b) reacting a nucleus, cytoplasm or mitochondria staining solution with an aptamer chip bound to the target cell; and (c) measuring the intensity of parts reacted with the cell staining solution.

Another purpose of the present invention is to provide a method for detecting a target protein using an aptamer chip, comprising (a) bringing an aptamer chip into contact with a sample comprising a target protein; and (b) reacting Coomassie Brilliant Blue solution with an aptamer chip bound to the target protein.

Another purpose of the present invention is to provide a method for quantifying a target protein using an aptamer chip, comprising (a) bringing an aptamer chip into contact with a sample comprising a target protein; (b) reacting Coomassie Brilliant Blue solution with an aptamer chip bound to the target protein; and (c) measuring the intensity of parts reacted with the Coomassie Brilliant Blue solution.

Another purpose of the present invention is to provide a method for reusing an aptamer chip, comprising (a) bringing a sample comprising a target cell into contact with a chip fixed by disulfide bond; (b) detecting or quantifying the target cell by reacting a cell staining solution with the aptamer chip bound to the target cell; (c) separating the aptamer by applying disulfide bond reduction solution on the aptamer chip; and (d) refixing an aptamer molecule to the chip from which an aptamer has been separated.

Another purpose of the present invention is to provide a method for reusing an aptamer chip, comprising (a) bringing a sample comprising a target protein into contact with a chip fixed by a disulfide bond; (b) detecting or quantifying the target protein by reacting Coomassie Brilliant Blue solution with the aptamer chip bound to the target protein; (c) separating the aptamer by applying disulfide bond reduction solution on the aptamer chip; and (d) refixing an aptamer molecule to the chip from which an aptamer has been separated.

Another purpose of the present invention is to provide a reusable aptamer chip used in the method for reusing the aptamer chip.

### Advantageous Effects

The present invention is economical because the detection and quantification of a specific target protein or a cell expressing a specific target protein on the surface may be easily performed on an aptamer-based biosensor chip. Particularly, the present invention is economical because expensive equipment is not required, particularly in the case of detection and quantification of proteins, and detection and quantification of cells does not cost a lot because a common cell staining can be used and expensive equipment or chemicals are not required.

Additionally, the biosensor in the present invention uses an aptamer unlike most protein biosensors, and thus the detection limit and background signals are low. Moreover, the biosensor has detection ability with high sensitivity.

Particularly, in the case of introducing an aptamer to the surface by a disulfide bond, the chip can be reused by reducing the disulfide bond unlike a chip with an aptamer immobilized through a covalent bond using cross-linked bond agents. Therefore, additional savings can be expected. Accordingly, the aptamer chip in the present invention can be used for development of medicines and diagnosis of various diseases with low cost.

### DESCRIPTION OF DRAWINGS

FIG. 1 shows schematically the principle of an aptamer-based chip according the present invention. An aptamer with a thiol group at the 5′ end is bound to the glass plate coupled to a thiol group through disulfide bond.

FIG. 2 is the result of quantification and identification of His-tagged proteins by Coomassie Brilliant Blue staining and an aptamer-based chip according to the present invention. (A) is the result of concentration of the His-tagged target proteins. Thrombin is used as a control. (B) is the result of the signal intensity on the concentration of the His-tagged target proteins.

FIG. 3 is the result of detection and quantification of a target cell expressing a specific protein on the surface using DAPI staining and an aptamer-based chip according the present invention.

FIG. 4 is the result and the process of reusing an aptamer-based chip according the present invention.

### OPTIMAL MODES FOR THE INVENTION

As an aspect, the present invention is to provide a method for detecting a target cell using an aptamer chip, comprising (a) bringing a sample comprising a target cell into contact with an aptamer chip; and (b) reacting a cell staining solution with an aptamer chip bound to the target cell.

As used herein, “aptamer chip” is used synonymously with “aptamer array”, “aptamer-based chip”, and refers to a common name of minutely stacking things by attaching the aptamer with high density to a chosen location on a wide variety of a solid surfaces including a polymer board like glass with improved surface, silicon, and polypropylene. The aptamer can be immobilized on the board.

The type of the board can be used with no restriction as long as it is a solid board for manufacturing an aptamer chip used generally in the art, preferably glass, alumina, ceramics, carbon, gold, silver, copper, aluminum, compound semiconductor and silicon, and so on, most preferably, glass plate. The surface treatment can be performed on the board in order to facilitate to attach an aptamer molecule and immobilize the molecule. Additionally, the surface treatment can be performed while including a functional group for immobilizing an aptamer on the substrate surface of an aptamer chip. For example, the board can be reformed with an aldehyde group, a carboxyl group, an amine group or a thiol group. In case of a glass board or a semiconductor board, an amine group (e.g. —NH₃, —NH₂) or a thiol group can be formed by silane treatment. Moreover, in order for effective silane treatment, the process for making a hydroxyl group (—OH) can be performed before silane treatment. Most preferably, a thiol group can be a functional group. The board can be treated by silane for making a thiol group a functional group.

An exemplary embodiment of the present invention shows that the glass surface was treated by piranha solution and thus a hydroxyl group (—OH) was exposed on the surface, and a thiol group was formed on the surface as a functional group by MPTMS ((3-Mercaptopropyl)trimethoxysilane) treatment (Example 3).

As used herein, “aptamer” refers to a single-stranded nucleic acid (DNA, RNA or variant nucleic acid) with a stable tertiary structure in itself, and a material binding specifically to an analysis material detected in a sample. Thus the existence of the target protein in the sample can be examined by the bond. According to a general method for manufacturing an aptamer, an aptamer can be fabricated by modifying 5′ end or 3′ end of an oligonucleotide to a thiol group (—SH), carboxyl group (—COOH), hydroxyl group (—OH) or an amine group (—NH₂) for binding a functional group on the aptamer chip after determining and synthesizing sequence of the oligonucleotide which has selectively high coherence with the protein expressed on the surface of the target protein or the target cell to be confirmed.

In terms of purpose of the present invention, the aptamer can bond specifically to the membrane protein overexpressed on the surface of the target cell. Preferably, the aptamer can be RNA bound to a thiol group at the 5′ end, but is not limited thereto.

The aptamer in the present invention, which can bind specifically to the analysis material to be detected in a sample, can be an oligonucleotide binding specifically to the proteins expressed particularly on the surface of the target cell by using in-vitro selecting method, SELEX, in order for detection and/or quantification of the target cell. The SELEX is in-vitro selecting method for identifying a single strand DNA, RNA, oligonucleotide, which performs selective functions in a wide variety of forms. The SELEX enables a desirable aptamer to be identified from oligonucleotides with different sequences of 10¹⁵ random populations. Each oligonucleotide has unique tertiary structure, and repeated selection of an oligonucleotide with a desirable function (e.g. selective recognition to a specific protein) and oligonucleotide sequences selected by a traditional molecular biological method are amplified. Through these iterative selection and amplification processes, the oligonucleotides having a selective binding affinity for desired target molecules or transition states of their chemical process finally account for most of the population. Additionally, the sequence of each aptamer finally obtained by the processes is identified. A commercially available automated SELEX kit may also be used (e.g. Biomek 2000 pipetting robot, Beckman Coulter, USA).

The aptamer can be freely selected depending on properties of the target cell, and be a RNA aptamer binding specifically to the protein overexpressed in the target cell but is not limited thereto. Preferably, the protein is a cell membrane protein expressed in the cell membrane, and any aptamer can be used without limitation if the aptamer binds specifically to the cell membrane protein expressed particularly in the target cell. As an example of unrestricted aptamers, the aptamer can bind specifically to HER2 (human epidermal growth factor receptor 2) or PSMA (prostate-specific membrane antigen) protein. An exemplary embodiment of the present invention shows that an aptamer is manufactured by selecting a typical protein expressed from the target cell, HER2 or PSMA. The sequence of HER2 or PSMA can be obtained from a public database such as NCBI GenBank but is not limited thereto. The coherence between the cell membrane protein and a RNA aptamer (K_{d}=a few pM, about 10⁻¹² M) is the same or higher than the coherence between the protein and an antibody (K_{d}=about 10⁻⁵ to 10⁻¹² M). Therefore, the aptamer chip using the coherence between the cell membrane protein and the aptamer binding specifically to the protein is simple and can provide exceptional detectivity with selectivity and sensitivity.

An exemplary embodiment of the present invention shows whether the cell expressing the protein can be captured specifically by reacting with the aptamer chip manufactured by using the RNA aptamer binding specifically to HER2 protein which is well known to be overexpressed in breast cancer cells as a cell membrane protein or the PSMA protein which is well known to be overexpressed in prostate cancer cells.

The step of bringing an aptamer chip into contact with a sample including the target cell in the step (a) is for detecting the target cell by capturing the target cell expressing the protein binding specifically to the aptamer.

As used herein, “target cell” refers to a cell which is attempted to be identified as existing in a sample by using an aptamer chip, particularly, a cell which can be detected by being captured by the aptamer chip through the bond between the cell and the membrane protein after expressing the cell membrane protein binding specifically to the aptamer. As an example of the cell membrane protein, any protein which is well known to those skilled in the art can be used without restriction. To be more specific, the cell membrane protein is not limited thereto but in the case of a protein, the expression level of which is increased or decreased when a disease arises, the development of the disease can be diagnosed with using the aptamer chip of the present invention. An exemplary embodiment of the present invention shows that breast cancer cell line expressing HER2 or prostate cancer cell line expressing PSMA, as a target cell, was used.

As used herein, “sample” refers to tissues, cells, whole blood, serum, saliva, sputum, or cerebrospinal fluid which have the potential to include the target cell to be detected, but is not limited thereto.

The specific bond reaction between an aptamer and a target cell in a sample arises when bringing an aptamer-fixed board into contact with the sample containing the target cell. For example, the target cell can be captured on the aptamer chip by the formation of a complex through direct bond between the aptamer and a cell membrane protein overexpressed in the target cell.

An exemplary embodiment of the present invention shows that the specific bond reaction between an aptamer binding specifically to HER2 or PSMA protein immobilized on the board and a sample containing breast cancer cell line overexpressing HER2 protein or prostate cancer cell line overexpressing PSMA protein was performed (Example 5).

The step of reacting cell staining solution with the aptamer chip bound to the target cell in the step (b) is for detecting the target cell bound specifically to the aptamer with the naked eye.

The cell staining solution of the present invention can be a cell nucleus and cytoplasm used for cell staining, or a material used for mitochondria staining. Preferably, DAPI (4′,6-diamidino-2-phenylindole), methylene blue, acetocarmine, toluidine blue, hematoxylin, or Hoechst, and so on, which are staining a cell nucleus, eosin, crystal violet, or orange G, and so on, which are staining cytoplasm, rhodamine 123, MitoTracker, Janus green B, tetrazolium salt, DASPMI (Dimethylaminostyrylmethylpyridiniumiodine), DASPEI (2-(4-(dimethylamino)styryl)-N-Ethylpyridinium Iodide), DiOC6 (3,3′-dihexyloxacarbocyanine iodide), DiOC7 (3,3′-Diheptyloxacarbocyanine Iodide), or JC-1 (5,5,6,6-tetrachloro-1,1,3,3-tetraethyl-benzimidazolylcarbocyanine chloride, and so on, which are staining mitochondria, can be used but are not limited thereto. Preferably, DAPI staining a cell nucleus can be used. The detection using cell staining solution is simple, and requires short time, and is performed with the naked eye under a simple light source. Therefore, when compared to the pre-existing detection methods for an aptamer chip such as, (i) an electrochemical method which detects the level of electron transmission before and after the bond between an aptamer and a target molecule, (ii) an optical method which is performed by measuring fluorescence intensity after labeling fluorescent materials, and (iii) mass spectrometry which analyzes and measures mass difference before and after the bond between an aptamer and a target material, the detection method using cell staining solution can simplify complex processes and reduce high costs because the method does not requires expensive equipment. Particularly, the aptamer detection using the existing fluorescence method requires the coupling of a fluorescent material to an aptamer itself or a complementary DNA binding to an aptamer, but the staining solution in the present invention can stain in a simple way in which reacting an aptamer with a sample for a few minutes after mixing an aptamer with the sample is made.

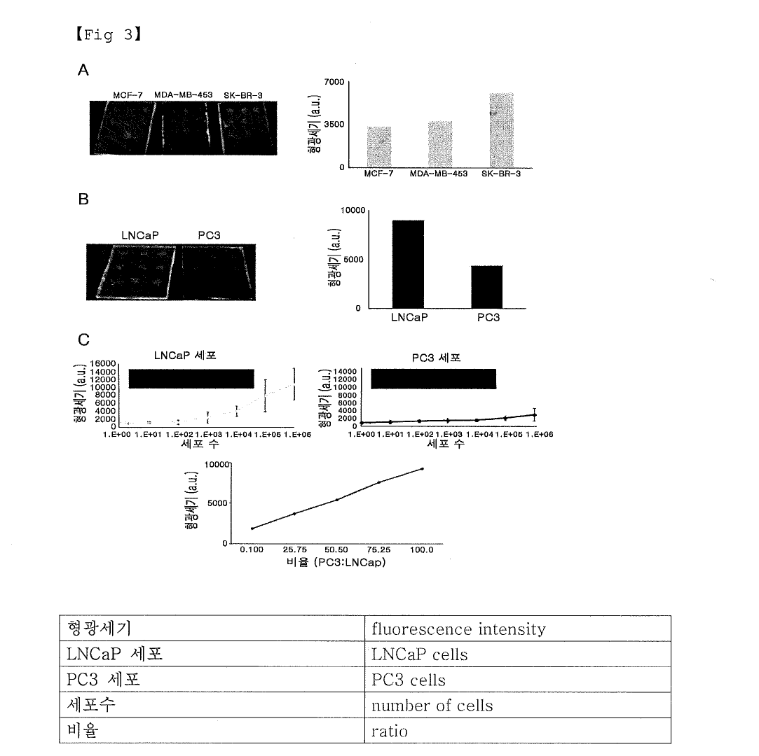

An exemplary embodiment of the present invention shows that after incubating a cell nucleus staining solution, DAPI, with a chip bound to a target cell at 37° C. for 15 min, the inventors examined with the naked eye and found that the target cell was bound specifically to a RNA aptamer by being exposed to UV. Additionally, the number of the target cells can be detected 10³, more preferably, 10⁴ (FIG. 3C) and thus the sensitivity was found to be high. Moreover, the detection ability was proportional to the ratio of the target cells in a sample in which the target cell was mixed with other cells (FIG. 3C). Therefore, it was found that the aptamer chip of the present invention could detect the target cell with high sensitivity and specificity.

As another aspect, the present invention is a method for quantifying a target cell using an aptamer chip, comprising (a) bringing a sample comprising a target cell into contact with an aptamer chip; (b) reacting a cell staining solution with an aptamer chip bound to the target cell; and (c) measuring the intensity of parts reacted with the cell staining solution.

The steps (a) and (b) are the same as the method for detecting a target cell described above.

The step of measuring the intensity of parts reacted with cell staining solution in the step (c) is for quantifying the target cell in the sample.

The target cell can be quantified because as the target cell concentration in the sample is increased, the signal intensity by reaction with cell staining solution is increased. A general optical scanner, a densitometry, an image analysis device, or Adobe Photoshop software can be used but is not limited thereto.

An exemplary embodiment of the present invention shows that it was found that as the target cell concentration is increased, fluorescence intensity by DAPI staining the cell nucleus is increased, and a wide range of 10³ to 10⁶ cells can be quantified (FIG. 3C).

As another aspect, the present invention is a method for detecting a target protein using an aptamer chip, comprising (a) bringing an aptamer chip into contact with a sample comprising a target protein; and (b) reacting Coomassie Brilliant Blue solution with an aptamer chip bound to the target protein.

The aptamer chip and the aptamer of the present invention is the same as defined above.

In terms of purpose of the present invention, the aptamer can bind specifically to a target protein. Preferably, the aptamer can be a RNA bound to a thiol group at the 5′ end but not limited thereto.

The aptamer can be selected freely depending on the target protein, and preferably, the aptamer can be a RNA aptamer binding specifically to oligohistidine but not limited thereto. The oligohistidine is serial histidine residues, but is not limited thereto, and is a general histidine-tag. Preferably, the oligohistidine can be 6 serial histidine residues.

In general, the binding force between a RNA aptamer and oligohistidine is Kd=˜3.78 pM higher than, or the same as the binding force between a protein and an antibody, Kd=˜10⁻⁵˜10⁻¹² M. Therefore, the binding force with a protein can be easily examined.

An exemplary embodiment of the present invention shows that it was examined whether a chip with a RNA aptamer binding specifically to oligohistidine was bound specifically to His-tagged thoredoxin, part of a His-tagged protein, FAF1 UBX.

The step of bringing an aptamer chip into contact with a sample containing the target protein in the step (a) is for capturing and detecting the target protein binding specifically to the aptamer.

As used herein, “target protein” refers to a material which is attempted to be detected in a sample using an aptamer chip. An example of the target protein is a general material used in the art and there is no limit. In more detail, the target protein derived from an organism the like, or a thing manufactured in vitro. For example, enzymes, antibodies, antigens, peptides, proteins derived from a microorganism, and proteins derived from animal and plant cells and organs can be the target proteins. Preferably, the target protein can be labeled by an oligohistidine, but is not limited thereto.

As used herein, “sample” refers to tissues, cells, whole blood, serum, saliva, sputum, or cerebrospinal fluid which have the potential to include the target protein to be detected, but is not limited thereto.

The specific bond reaction between an aptamer and a target protein in a sample arises when bringing an aptamer-fixed board into contact with the sample containing the target protein. For example, the formation of a complex through direct bond between the aptamer and the target protein can arise, or the combination containing reactions of the aptamer transformation or modification by enzymatic reaction of the protein in the sample can arise.

An exemplary embodiment of the present invention shows a specific binding by bringing an aptamer immobilized on the board into contact with a sample containing a target protein with complementary sequence (Example 4).

The step of reacting Coomassie Brilliant Blue solution with the aptamer chip bound to the target protein in the step (b) is for detecting the target protein bound specifically to the aptamer with the naked eye.

The Coomassie Brilliant Blue solution of the present invention is generally used in staining proteins and binds to proteins through 6 phenyl groups and 2 sulfonic groups in the Coomassie Brilliant Blue. As a result of research for overcoming the high costs of methods such as the (i) electrochemical method which detects the level of electron transmission before and after the bond between an aptamer and a target molecule, (ii) optical method which is performed by measuring fluorescence intensity after labeling fluorescent materials, and (iii) mass spectrometry which analyses and measures mass difference before and after the bond between an aptamer and a target material, the inventors developed the above method for the first time, which is for detecting and quantifying specifically the target protein with the naked eye through binding the target protein to Coomassie Brilliant Blue solution. The staining with Coomassie Brilliant Blue solution can be examined with the naked eye without additional equipment.

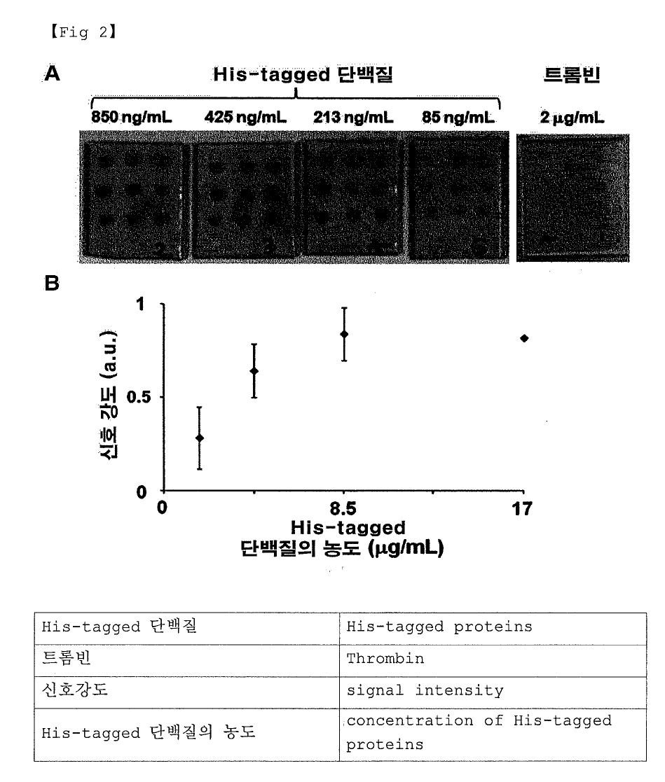

An exemplary embodiment of the present invention shows that it was found with the naked eye that the target protein bound specifically to a RNA aptamer after reacting Coomassie Brilliant Blue solution with a chip bound to proteins at 4° C. for 1 hour. Additionally, it was found that the detection sensitivity of the target protein, 85 ng/ml, is high (FIG. 2A).

As another aspect, the present invention is a method for quantifying a target protein using an aptamer chip, comprising (a) bringing an aptamer chip into contact with a sample comprising a target protein; (b) reacting Coomassie Brilliant Blue solution with an aptamer chip bound to the target protein; and (c) measuring the intensity of parts reacted with the Coomassie Brilliant Blue solution.

The steps (a) and (b) are the same as the method for detecting a target protein described above.

The step of measuring the intensity of parts reacted with Coomassie Brilliant Blue solution in the step (c) is for quantifying the target protein in the sample.

The target protein can be quantified because as the target protein concentration is increased, the intensity by reaction with Coomassie Brilliant Blue solution is increased. A general optical scanner, a densitometry, an image analysis device, or Adobe Photoshop software can be used but is not limited thereto.

An exemplary embodiment of the present invention shows that it was found that as the target protein concentration is increased, Coomassie Brilliant Blue intensity is increased, and a wide range of 85 ng/ml to 425 ng/ml can be quantified (FIGS. 2A and B).

As another aspect, the present invention is a method for reusing an aptamer chip, comprising (a) bringing a sample comprising a target cell into contact with a chip having the aptamer fixed by disulfide bond; (b) detecting or quantifying the target cell by reacting a cell staining solution with the aptamer chip bound to the target cell; (c) separating the aptamer by treating disulfide bond reduction solution on the aptamer chip; and (d) refixing an aptamer molecule to the chip from which an aptamer is separated.

Reusing the aptamer chip refers to the use of an aptamer chip more than once, and the number of reuses of the chip are not limited. Additionally, the chip can be used continuously as long as reusing the chip does not affect the detection intensity.

The steps (a) and (b) are the same as the method for detecting and quantifying the target cell described above other than immobilizing the aptamer on the board through disulfide bond.

The aptamer chip reused in the present invention can be preferably a RNA aptamer with a thiol group (—SH) at the 5′ end.

The board of the aptamer chip can be any one selected from the group consisting of glass, alumina, ceramics, carbon, gold, silver, copper, aluminum, and silicon, but is not limited thereto.

The board of the aptamer chip can preferably have a thiol group as a functional group on the surface. The board can be treated by silane in order to have a thiol group as a functional group. The board manufactured by the method can bind a RNA aptamer with a thiol group at the 5′ end through the thiol group by disulfide bond. The disulfide bond makes reuse of the chip by reduction possible, unlike a chip with an aptamer immobilized through covalent bond using pre-existing cross-linked bond agents.

The agent reducing the disulfide bond in the step (c) can be used without restriction. For example, DTT (dithiothreitol), β-mercaptoethanol, β-mercaptoethylamine, or TCEP (tris[2-carboxyethyl]phosphine) can be the agent. Preferably, DTT can be the agent.

In the case of the aptamer chip used for detecting the target cell, preferably, dissociating the target cell bound to the aptamer chip can be added by treatment of a cell lysis detergent before the step (c). Dissociating the target cell can be performed by cell lysis by the cell lysis detergent. Any material as a cell lysis detergent can be used without restriction if the material dissociates the cell from the aptamer chip by cell lysis. Commercial products, preferably, like triton X-100, tween 20, span 20, SDS (sodium dodecyl sulfate) can be easily used. To be more specific, the cell lysis detergent diluted at the appropriate level concentration can be applied for a constant time. An exemplary embodiment of the present invention shows that the aptamer chip bound to the target cell was treated with 1% triton X-100 (PBS solution) for 1 hour.

As another aspect, the present invention is a method for reusing an aptamer chip, comprising (a) bringing a sample comprising a target protein into contact with a chip having the aptamer fixed by disulfide bond; (b) detecting or quantifying the target protein by reacting Coomassie Brilliant Blue solution with the aptamer chip bound to the target protein; (c) separating the aptamer by applying a disulfide bond reduction solution on the aptamer chip; and (d) refixing an aptamer molecule to the chip from which an aptamer is separated.

The steps (a) and (b) are the same as the method for detecting and quantifying the target protein described above other than immobilizing the aptamer on the board through a disulfide bond.

In the present invention, the definition and the principle of reusing an aptamer chip, an aptamer used for an aptamer chip available to reuse, and a method for producing the aptamer are described above.

The agent reducing the disulfide bond in the step (c) can be used without restriction, and the examples are as described above.

In the case of the aptamer chip used for detecting the target protein, preferably, dissociating the target protein bound to the aptamer chip can be added by treatment of urea before the step (c). Dissociating the target protein can be performed by protein denaturation by urea.

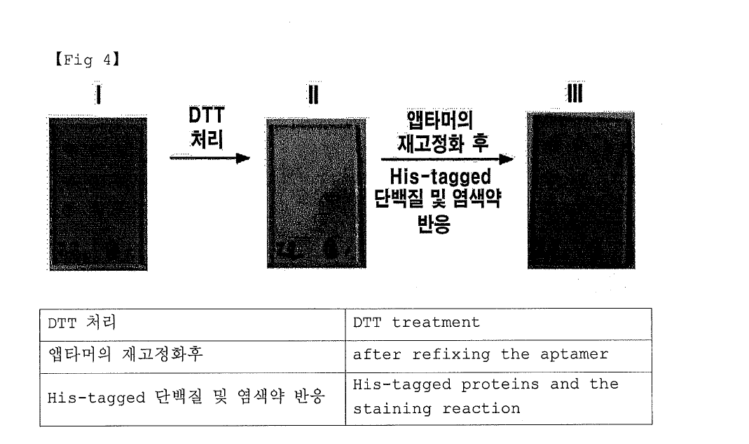

An exemplary embodiment of the present invention shows that proteins were modified by reacting an aptamer chip with 8M urea, and then the aptamer was removed through reducing disulfide bond by DTT treatment, and then His-tagged proteins were reacted with the aptamer chip remanufactured by immobilizing a RNA aptamer, which was introduced to 5′ thiol group, on the chip. Consequently, as it was found that there was a staining intensity similar to that in the first experiment (FIG. 4), the chip and the method for reusing the chip can be stably used.

As another aspect, the present invention provides an aptamer chip where an aptamer specifically bound to the target cell or the target protein provided from the method for reusing the aptamer chip is combined with a board by disulfide bond.

Description of the aptamer chip is the same as described above.

### DETAILED DESCRIPTION OF THE EMBODIMENTS

Hereinafter, the present invention will be described in detail through examples. However, the examples are only individual examples of the present invention and the present invention is not limited thereto.

### Example 1

### Preparing Reagents and Equipment

Chemicals used in the present invention were purchased from supplier companies, and used without additional purification. Deionized water treated by DEPC (diethyl pyrocarbonate) was used in all experiments. UV absorption was measured via Agilent 8453 UV-Visible spectrometer. All experiments were performed two times.

### Example 2

### Manufacturing an Aptamer

2-1. Manufacturing an Aptamer for Detecting His-Tagged Proteins

In order to bind an aptamer selectively to His-tagged proteins, a RNA aptamer with a thiol group at the 5′ end is manufactured by the following method.

The RNA aptamer combining specifically with His-tagged proteins was synthesized using complementary oligonucleotide (5′-GCCAG CTCCC GGGGC CAATC CCAAC CAGAC CACCC ATAGC CCCCC CTATA GTGAG TCGTA TTAGT CC-3′, Sequence ID NO 1) containing T7 promotor sequence at the 5′ end. The synthesized RNA aptamer sequence is as follows:

Sequence ID NO 2:
5′-GCUAU GGGUG GUCUG GUUGG GAUUG GCCCC GGGAG
CUGGC-3′.

The synthesized RNA aptamer was attached to a thiol group (—SH) at the 5′ end using the method provided by shin et al., and Kim et al. (shin et al., Bioorg. Med. CHem. Lett., 2010, 20: 3322-3325; Kim et al., Tet. Lett., 2010, 51: 3446-3448). To be more specific, 5′ end was modified to a thiol group using an enzyme in order to introduce a thiol group to 5′ end of the RNA aptamer. For efficient transcription, GSMP 5′-deoxy-5′-thioguanosine-5′-monophosphaorohioate was synthesized as a substrate for T7 RNA polymerase requiring guanosine (Kim et al., Tet. Lett., 2010, 51: 3446-3448). A thiol group was introduced to 5′ end of the RNA molecule through alkaline phosphatase treatment after in vitro transcription.

2-2. Manufacturing an Aptamer for Detecting a Cell Expressing HER2 (Human Epidermal Growth Factor Receptor 2) or PSMA (Prostate-Specific Membrane Antigen) on the Surface

An aptamer with a thiol group at the 5′ end was manufactured by the following method, which binds specifically to a cell expressing HER2 (human epidermal growth factor receptor 2) or PSMA (prostate-specific membrane antigen) on the surface.

First, the RNA aptamer binding specifically to HER2 proteins at the 3′ end was synthesized as 34-mers (5′-AGC CGC GAG GGG AGG GAU AGG GUA GGG CGC GGC U-3′ Sequence ID NO 5) and 82-mers (5′-GGG ACG CGU GGU ACC AAA AGU UGU GAG GGG AGG GAU AGG GUA GGG CAC GAC UAG UCA AGA AAA UGA AGC UUC CGC GGG GAU C-3′ Sequence ID NO 6), using complementary oligonucleotide sequence (5′-AGC CGC GCC CTA CCC TAT CCC TCC CCT CGC GGC TCC CCT ATA GTG AGT CGT ATT AGT CC-3′ Sequence ID NO 3; 5′-GAT CCC CGC GGA AGC TTC ATT TTC TTG ACT AGT CGT GCC CTA CCC TAT CCC TCC CCT CAC AAC TTT TGG TAC CAC GCG TCC CCC CTA TAG TGA GTC GTA TTA GTC C-3′ Sequence ID NO 4) containing T7 promotor sequence, and the method provided by Shin et al., and Kim et al. (Shin et al., Bioorg. Med. Chem. Lett., 2010, 20: 3322-3325; Kim et al., Tet. Lett., 2010, 51: 3446-3448). Meanwhile, the RNA aptamer binding specifically to PSMA proteins at the 3′ end was synthesized as 71-mers (5′-GGG AGG ACG AUG CGG AUC AGC CAU GUU UAC GUC ACU CCU UGU CAA UCC UCA UCG GCA GAC GAC UCG CCC GA-3′ Sequence ID NO 8; Biomaterials, 2011, 32: 2124-2132), using complementary oligonucleotide sequence (5′-TCG GGC GAG TCG TCT GCC GAT GAG GAT TGA CAA GGA GTG ACG TAA ACA TGG CTG ATC CGC ATC GTC CTC CCC CCT ATA GTG AGT CGT ATT AGT CC-3′ Sequence ID NO 7) containing T7 promotor sequence, and the method provided by Shin et al., and Kim et al. (Shin et al., Bioorg. Med. Chem. Lett., 2010, 20: 3322-3325; Kim et al., Tet. Lett., 2010, 51: 3446-3448). The 5′ end of the RNA aptamer was modified to a thiol group through introducing a sulfhydryl group by an enzyme. For efficient transcription, GSMP (5′-deoxy-5′-thioguanosine-5′-monophosphaorohioate) was synthesized as a substrate for T7 RNA polymerase requiring guanosine (Kim et al., Tet. Lett., 2010, 51: 3446-3448). A thiol group was introduced to 5′ end of the RNA molecule through alkaline phosphatase treatment after in vitro transcription.

### Example 3

### Manufacturing an Aptamer Chip

An aptamer chip was prepared by applying the pre-existing method in another thesis (Lee and Hah, Bioorg. Med. Chem. Lett., 2012, 22: 1520-1522; Chen et al., Biosensors and Bioelectronics, 2007, 22: 926-932).

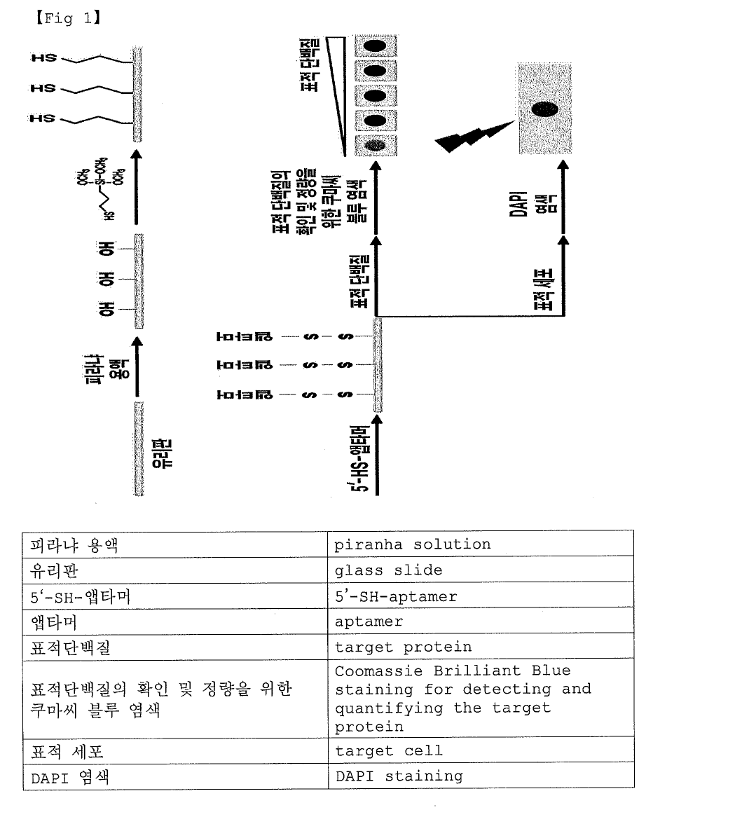

Briefly, the surface of a glass slide (1.5 cm×2 cm) was cleaned by treatment of piranha solution (when the whole piranha solution is 1, 30% H₂O₂ solution is 0.3, H₂SO₄ solution is 0.7; Stavyiannoudaki et al., Anal. Bioanal. Chem., 2009, 395: 429-435) at 80° C. for 1 hour. Then, the slide was washed repeatedly by deionized water at room temperature, and then sonication was performed on the slide for 20 minutes. Additionally, after desiccating the glass slide at 100° C. oven for 1 hour, the glass slide was immersed in ethanol solution (MPTMS:EtOH=1:20) treated by MPTMS ((3-mercaptoptopyl)trimethoxysilane) for introducing a thiol group on the surface. After 15 minutes, 600 μl of 0.01 M NaOH aqueous solution was put in ethanol solution treated by MPTMS and then maintained for 30 minutes. Moreover, the glass slide was washed repeatedly by deionized water and ethanol in order. The desiccating step was performed at room temperature before additional modification. 1 μl of RNA solution (aptamer) with a thiol group at the 5′ end was spotted on the glass slide with a thiol group on the surface, and then reacted with each other for making disulfide bond at 4° C. for more than 12 hours (overnight). Next, RNAs not reacted with the glass slide were removed repeatedly using PBS solution (pH 7.4) (FIG. 1).

### Example 4

### Quantifying and Identifying a Target Protein Binding to an Aptamer Chip Through Coomassie Brilliant Blue Staining

For the selectivity and the sensitivity to His-tagged protein in an aptamer-based chip, the inventors used His-tagged thoredoxin of purified recombinant human FAF1 (Fas-associated factor1) UBX (Ultrabithorax) (Shin et al., Bioorg. Med. CHem. Lett., 2010, 20: 3322-3325). FAF1 UBX is well known to bind well to N-terminal of p97/VCP (AAA+, one of ATPase, used in various cell processes like nuclear envelope reconstruction process, cell cycle, golgi formation of post mitochondria, and prohibiting apoptosis). His-tagged protein samples were overexpressed, and purified by the pre-existing well-known method in the former thesis. His-tagged protein solution was cultured at 4° C. for 3 hours with the aptamer chip manufactured in the example 3. After that, the chip was washed 3 times using PBS solution (pH 7.4) for removing proteins not reacted with the chip. Thrombin, which is not His-tagged, was used as a control.

Coomassie Brilliant Blue staining was used for the detection and quantification of the target protein binding to the aptamer-based chip. The chip binding to the protein was reacted at 4° C. for 1 hour with Coomassie Brilliant Blue solution (Coomassie Brilliant Blue R-250 staining solution, Bio-Rad). Then, the chip was washed 3 times using destaining solution (EtOH:H₂O:AcOH=45:45:10), and additionally was washed using PBS. The signal gained from the photograph of the result was quantified using Gel-Pro Analyzer (Media Cybernetics).

Consequently, low His-tagged protein concentration, 85 ng/ml, was detected within 1 hour in reaction time (FIG. 2A). Additionally, we examined that the method of the present invention can quantify a wide range of the signal, 85 ng/ml to 425 ng/ml (FIG. 28). Moreover, the binding was examined with the naked eye. As a control, thrombin, which is not His-tagged, bound at higher level of concentration 2 μg/ml, but the result of Coomassie Brilliant Blue staining was not shown. Therefore, we examined that only the target protein selectively bound to the aptamer chip.

The result supports that the method of the present invention is able to detect effectively the target protein at high level of the sensitivity with the naked eye using the aptamer chip as well as to quantify information with simple equipment.

### Example 5

### Quantification and Detection of a Target Cell Bound to an Aptamer Chip Through DAPI Staining

For examining the cell specific selectivity and the cell specific sensitivity of the aptamer-based chip manufactured in the example 3, the inventors incubated well-mixed cell suspension at 4° C. for 30 minutes for minimizing non-specific binding, and after putting the aptamer chip in the cell suspension, those were incubated at 37° C. for 1 hour. After that, the chip was washed 3 times using PBS for removing non-binding cells, and then cell permeabilization was made by 0.1% triton X-100 (PBS solution) treatment for 5 minutes, and additional washing was made 3 times using PBS. The staining was performed using DAPI solution with 2 μg/ml concentration at 37° C. for 15 minutes, and then was washed using PBS, and then the signal by being exposed to UV (365 nm) was detected. The fluorescent signal was digitized using a spectrophotometer for quantification.

First, 6 cancer cell lines (SK-BR-3, MDA-MB-453, MCF-7, LNCaP, PC3, and HeLa), which have different level of HER2 expression, were cultured and then the cell lines with the same quantity was applied to the aptamer chip in which a RNA aptamer bound selectively to HER2 proteins overexpressed in breast cancer cells was fixed. As described above, the aptamer chip was incubated at 37° C. for 1 hour, and then was washed 3 times using PBS and thus non-binding cells were removed. DAPI staining generally used in cell nucleus staining was used for detecting and quantifying cells binding to the aptamer chip reacted with the cells. The result is shown in FIG. 3A. 10⁴ breast cancer cells (SK-BR-3, MDA-MB-453, and MCF-7) were successfully detected within 1 hour of incubation time. DAPI signals were not detected in the aptamer chip to which prostate cancer cells (LNCaP and PC3) and cervical cancer cells (HeLa) not expressing HER2 proteins were applied.

Additionally, the experiments of prostate cancer cells, LNCap and PC3, which have different level of PSMA expression, were done in the same way, using an aptamer chip in which a RNA aptamer bound selectively to PSMA proteins overexpressed in prostate cancer cells was fixed. The result is shown in FIG. 3B. The strong fluorescent signal by DAPI in LNCap overexpressing PSMA was detected but only a weak signal in PC3 nearly not expressing PSMA was detected.

Further, after mixing prostate cancer cells, which have different level of PSMA expression, with various ratios, those were applied to the aptamer chip. After that, the signal by DAPI staining was detected. As shown in FIG. 3C, LNCap cells overexpressing PSMA showed that as the number of applied cells was increased, the fluorescent signal was increased, but PC3 cells not expressing PSMA showed that the signal was not increased although the number of cells was increased by 10⁶. It was found that as the LNCap ratio was increased, the fluorescent signal was increased in the mixed sample.

Therefore, the cell detection method of the present invention has been shown to be able to detect efficiently even a low level of 10³ target cells by selecting a desirable aptamer. Additionally, the method was able to detect efficiently only the target cell proportional to mixing ratio in a mixed sample, and showed high sensitivity and the high specificity.

### Example 6

### Examining Reusability of an Aptamer Chip

For examining the reusability of an aptamer chip of the present invention, the aptamer chip treated by staining processes in the example 4 was reacted with 8M urea at 40° C. for 2 minutes. Meanwhile, the aptamer chip processed by the staining and the detection in the example 5 was treated by 1% triton X-100 (PBS solution) for 1 hour. After that, the aptamer chip was treated by DTT (dithiothreitol) for reducing disulfide bond, and then was washed 3 times using PBS solution (pH 7.4). Then, a RNA aptamer with a thiol group at the 5′ end was immobilized on the chip. The reformed aptamer chip was reacted with His-tagged proteins, HER2, or PSAM again.

As a result, the staining intensity was similar to the first experiment where the aptamer chip was used (FIG. 4).

The result supports that the chip can be reused several times while maintaining the chip functions because the aptamer, from which the target protein was dissociated through modifying the complex of the aptamer and the target protein by urea, can be collected. Additionally, in the case of introducing an aptamer to the surface of a chip by disulfide bond, the result supports that unlike a aptamer chip with an aptamer immobilized through covalent bond using cross-linked bond agents, the chip can be reused through reducing the disulfide bond, and costs can be reduced and the present invention can provide an aptamer chip with high reproducibility and the high reusability.

1. A method for detecting and/or quantifying a target cell using an aptamer chip, comprising:
(a) bringing a sample comprising a target cell into contact with an aptamer chip; and
(b) reacting a cell staining solution with an aptamer chip bound to the target cell.

2. The method of claim 1, further comprising measuring the intensity of parts reacted with the cell staining solution.

3. A method for detecting and/or quantifying a target protein using an aptamer chip, comprising:
(a) bringing an aptamer chip into contact with a sample comprising a target protein; and
(b) reacting Coomassie Brilliant Blue solution with an aptamer chip bound to the target protein.

4. The method of claim 3, further comprising measuring the intensity of parts reacted with the Coomassie Brilliant Blue solution.

5. The method of claim 1, wherein the detection of the target cell is distinguished with the naked eye by being exposed to UV.

6. The method of claim 1, wherein the cell staining solution is selected by the group consisting of DAPI (4′,6-diamidino-2-phenylindole), methylene blue, acetocarmine, toluidine blue, hematoxylin, and Hoechst, which are staining cell nucleus; or eosin, crystal violet or orange G, which are staining cytoplasm; and rhodamine 123, MitoTracker, janus green B, tetrazolium salt, DASPMI (Dimethylaminostyrylmethylpyridiniumiodine), DASPEI (2-(4-(dimethylamino)styryl)-N-Ethylpyridinium Iodide), DiOC6 (3,3′-dihexyloxacarbocyanine iodide), DiOC7 (3,3′-Diheptyloxacarbocyanine Iodide) and JC-1 (5,5,6,6-tetrachloro-1,1,3,3-tetraethyl-benzimidazolylcarbocyanine chloride), which are staining mitochondria.

7. The method of claim 3, wherein the detection of the target protein is measured with the naked eye.

8. The method of claim 1, wherein the aptamer is a RNA aptamer combined with a thiol group (—SH) at the 5′ end.

9. The method of claim 1, wherein the aptamer is bound specifically to HER2 (human epidermal growth factor receptor 2), or PSMA (prostate-specific membrane antigen).

10. The method of claim 1, wherein the target cell in the step (a) is a cell expressing HER2 or PSMA.

11. The method of claim 3, wherein the aptamer is combined specifically with an oligohistidine.

12. The method of claim 3, wherein the target protein in the step (a) is labeled by an oligohistidine.

13-14. (canceled)

15. A method for reusing an aptamer chip, comprising:
(a) bringing a sample comprising a target cell into contact with a chip having the aptamer fixed by disulfide bond;
(b) detecting or quantifying the target cell by reacting a cell staining solution with the aptamer chip bound to the target cell;
(c) separating the aptamer by treating disulfide bond reduction solution on the aptamer chip; and
(d) refixing an aptamer molecule to the chip from which an aptamer is separated.

16. The method of claim 15, wherein a step of separating a target cell bound an aptamer chip through treatment of cell lysis detergent is added before the step (c).

17. A method for reusing an aptamer chip, comprising:
(a) bringing a sample comprising a target protein into contact with a chip having the aptamer fixed by a disulfide bond;
(b) detecting or quantifying the target protein by reacting Coomassie Brilliant Blue solution with the aptamer chip bound to the target protein;
(c) separating the aptamer by treating disulfide bond reduction solution on the aptamer chip; and
(d) refixing an aptamer molecule to the chip from which an aptamer is separated.

18. The method of claim 17, wherein a step of separating a target protein through application of urea is added before the step (c).

19. The method of claim 15, wherein the aptamer is a RNA aptamer combined with a thiol group (—SH) at the 5′ end.

20-21. (canceled)

22. The method of claim 3, wherein the aptamer is a RNA aptamer combined with a thiol group (—SH) at the 5′ end.

23. The method of claim 3, wherein the aptamer is bound specifically to HER2 (human epidermal growth factor receptor 2), or PSMA (prostate-specific membrane antigen).

24. The method of claim 17, wherein the aptamer is a RNA aptamer combined with a thiol group (—SH) at the 5′ end.
